(19) United States
(12) Patent Application Publication
(10) Pub. No.: US 2018/0271774 A1
(43) Pub. Date: Sep. 27, 2018

(54) INFUSION OBTAINED FROM A MIXTURE OF SPECIES OF THE SIDERITIS PLANT FOR TOPICAL CUTANEOUS OR OTHER USE AND PRODUCTION METHOD THEREFOR

(71) Applicant: APIVITA S.A., Markopoulo (GR)

(72) Inventors: Nikolaos KOUTSIANAS, Koropi (GR); Sofia LETSIOU, GRENOBLE (GR); Konstantinos GARDIKIS, Athens (GR); Anna PATERA, Athens (GR); Panagiota DRAGANI, Athens (GR)

(21) Appl. No.: 15/764,118

(22) PCT Filed: Sep. 12, 2016
(86) PCT No.: PCT/GR2016/000043

§ 371 Date: Mar. 28, 2018

(30) Foreign Application Priority Data
Sep. 28, 2015 (GR) 20150100418

(51) Int. Cl. A61K 8/9789; A61Q 19/08

(52) U.S. Cl. CPC A61K 8/9789; A61Q 19/08

(57) ABSTRACT

Infusion from a mixture of species of the Sideritis plant for topical cutaneous or other uses formed by mixing the plants of the Sideritis family with the names Sideritis perfoliata/Sideritis scardica/Sideritis raeseri at proportions by weight from 70/25/5 to 40/50/10, with deionised water as extraction solvent, at a proportion by weight of the mixture of the Sideritis plants to the solvent from 0.01% to 10% and used either as such for topical cutaneous use, or as a component of cosmetic or cosmeceutical products at a proportion by weight from 0.01% to 80% in the produced final product, which is produced by an extraction device in which the pressure and temperature are controlled where the plants are placed in comminuted form and the solvent is added and the extraction procedure takes place under a pressure of 0 bar to 10 bar at 37° C. to 98° C., for from one to thirty minutes.

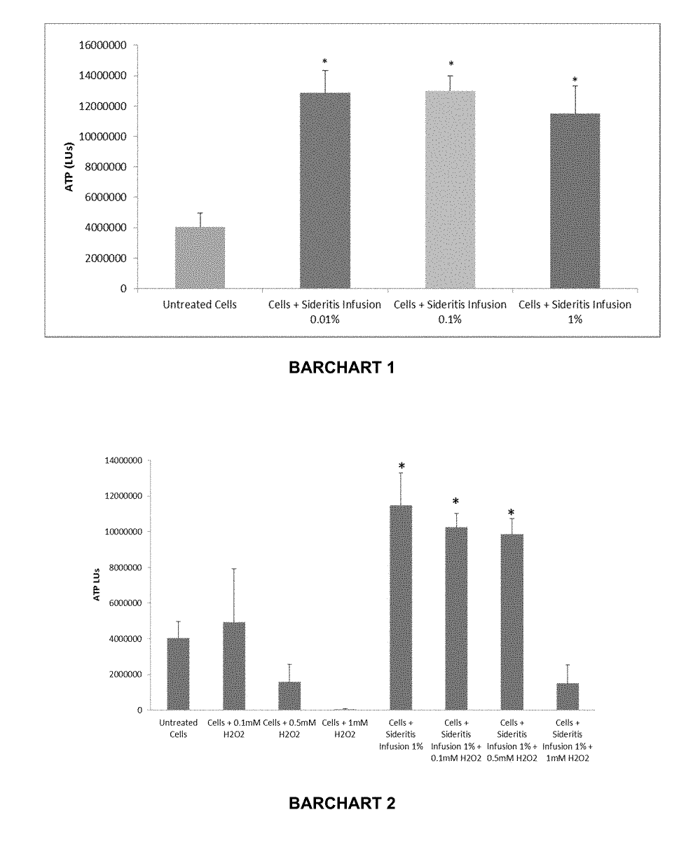

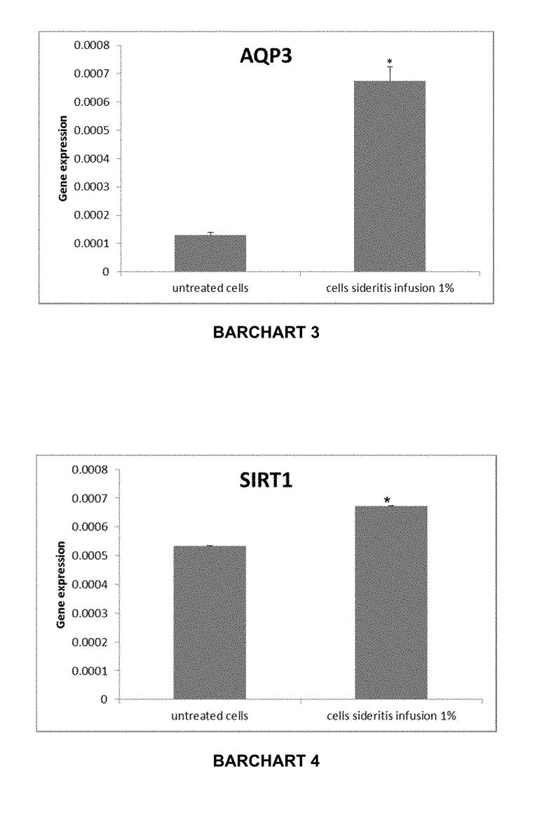

### TECHNICAL FIELD

The present invention relates to an infusion obtained from a mixture of various species of the Sideritis plant, which is characterized in that it is intended for topical cutaneous administration or as a component of cosmetic or cosmeceutical products, its use combining antioxidant properties, anti-aging properties, protective properties against cellular aging, moisturizing properties and photoprotective properties.

### TECHNICAL BACKGROUND

Until now, no such product has been mentioned worldwide. In particular, the genus of the Sideritis plant includes more than 150 species which occur in temperate and tropical climates of the Northern Hemisphere. In Greece, 10 taxa are found in 7 species of the genus. Its aerial parts are used traditionally in Spain, Turkey and Greece as beverages and are known as “mountain tea”. Modern studies validate the traditional use of the plants of the specific genus, since it was found that the extracts and essential oils present antioxidant and anti-inflammatory activity as well as antimicrobial activity. For example, anti-HIV, analgesic, inhibitory actions etc. may be mentioned. The pharmaceutical action of the species of the Sideritis genus is due to the chemical ingredients that have been discovered, such as terpenes, flavonoids, coumarins, sterols, iridoids, lignans and essential oils.

In bibliography, the antioxidant action of the species of the Sideritis plant is mainly described. The most important antioxidant components of Sideritis are the flavonoids such as the 3-and 8-disubstituted flavones, glycosidic flavonoids, aglycal flavonoids, poly-hydroxy-flavones and phenylpropanate esters of flavonoids. Due to the presence of the above components, the extracts of the Sideritis plants are considered to have a moderate to strong antioxidant action, depending on the type of the plant, the harvest period, the drying method, the type of the plant and the extraction method.

Also, in bibliography, the action of extracts of the Sideritis plants as associated with neuroprotective (Gonzalez-Burgos et al.), antioxidant (Guvenc et al., Yumrutas et al.) and anti-inflammatory (Kupeli et al.) properties have been mentioned. Also, the variation of the cognitive functions (Feistel and Walbroel) and the improvement of the attention deficit-hyperactivity syndrome (Knorle and Schnierle) have been mentioned. The above properties are demonstrated by laboratory studies which either do not involve cells or involve non-cutaneous human cells, or relate to the systemic administration of extracts of the Sideritis plants.

On the contrary, until now the topical administration of Sideritis extracts has not been described in the bibliography, although it constitutes an attractive choice in light of safe use and efficiency.

The present invention aims at the preparation and use of a new infusion derived from specific species of Sideritis plants, which after topical administration presents substantial anti-aging action, action against cellular aging, antioxidant action, moisturizing action and photoprotective action.

### DISCLOSURE OF THE INVENTION

In order to make our invention understood by those skilled in the art, the method of producing the desired extract is described below.

According to the proposed embodiment of the invention, in order that the desired mixture of the Sideritis plants is produced, initially three different species of the Sideritis family, in particular Sideritis perfoliata/Sideritis scardica/Sideritis raeseri are mixed in proportions by weight 70/25/5 to 40/50/10. Subsequently, the species are thoroughly mixed and the plants are comminuted to pieces having dimensions 1-10 mm so that their contact surface to the extraction solvent is large as possible.

As extraction solvent, deionised water is used that is obtained by the reverse osmosis method according the following procedure:

Tap water is input in a raw water tank (2 m³), by a suitable pumping system, passed through an automatic turbidity filter in order to remove the turbidity and the solid particles and active carbon for the removal of chlorine and organic load, and then an antiscalant is dosed for binding its hardness. Before its input in the central assembly of the reverse-osmosis unit, it is passed through a 1-micron cartridge filter. Subsequently, after the water has been suitably treated for use, in reverse osmosis it is input in the reverse-osmosis unit having a throughput of 350 It/h with a recovery of 70%. The water produced in the unit is kept in a stainless steel tank having a volume of 5 m³. From this tank, the water is supplied by a suitable pumping assembly to the deionizer and is guided on line to the UV Sideritis extraction tank. In order to avoid stagnant water in the network, the water is continuously circulated returning to the tank.

The quality requirement for the deionised water is: <=1 μS/cm at 25° C., which meets the specification of the European Pharmacopoeia for the preparation of parenteral pharmaceutical products. This quality is necessary in order to generate stable infusions of the Sideritis species, as the presence of salts varies considerably the solubility and stability of the secondary metabolites of the plants, to which metabolites the biological action on human cutaneous cells is attributed.

The proportion of the mixture of the Sideritis plants to the solvent by weight is from 0.01% to 10%.

In order to perform the desired extraction, the following procedure is followed: the mixture of the Sideritis species is placed in a sack filter with pores of a diameter of 100μ. The sack filter is then placed in the extraction device where the pressure and temperature can be controlled. Thereafter, the solvent is added and the extraction procedure begins. The extraction takes place under pressure from 0 bar to 10 bar at 37° C. TO 98° C. The duration of the extraction may vary from 1 min to 30 min. Thereafter, different procedures are performed according to the use of the final product.

### Case 1. Use of the Infusion Directly on the Skin

After completion of the extraction procedure, the sack filter with the plant is removed from the device and the infusion is conditioned to a temperature of 20° C.-25° C. Thereafter, the plant is removed from the device and passed through cartridge filters of a pore diameter of 10μ-5μ-1μ-0.2μ and directly packaged. This product is suitable for topical administration.

### Case 2. Use as a Component of Cosmetic and Cosmeceutical Products

After completion of the extraction procedure, the sack filter with the plant is removed from the device and the infusion is conditioned to a temperature of 50° C. -70° C. Thereafter, the infusion is filtered through a sack filter having a pore diameter of 25μ and is directly used as an active component in the aqueous phase of cosmetic and cosmeceutical products. The proportion by weight of the infusion in the final form may be from after in cosmetic formulation carriers as an active component in proportion from 0.01% to 80%.

### EXAMPLE

Three different plant species of the Sideritis family and in particular Sideritis perfoliata/Sideritis scardica/Sideritis raeseri at proportions by weight 55/38/7 are mixed together and the plants are comminuted to pieces having dimensions of 10 mm. These are placed in a sack filter having a pore diameter of 100μ and the system is introduced in an extraction device. Deionised water obtained by the double reverse-osmosis method, specification: <=1 μS/cm at 25° C., is added and the extraction is performed under the following conditions:
Pressure=0 bar
Temperature=95° C.
Time=9 min

After completion of the extraction procedure, the sack filter is removed from the device and the infusion is conditioned to 70° C. Thereafter, it is filtrated through a sack filter having a pore diameter of 25μ and is used directly as an active component in the aqueous phase of a cosmetic product at proportion by weight 66% in the final formulation.

### Experimental Demonstration of the Action of the Infusion

In order to demonstrate the action of the infusion, its interaction with human dermal cells and in particular with the human NHDF cell line was studied. NHDF (primary Human Dermal Fibroblasts), derived from human primary normal dermal fibroblasts, that have been isolated from adult humans. The specific cell line was cultured in the proposed nutrient medium, FGM™-2 BulleKit™, which contains 2% serum, while the necessary conditions are: 95% humidity, 5% CO₂ and temperature 37° C.

The initial goal was to evaluate the cell viability/vitality of the dermal fibroblasts after 48 h incubation of the infusion at three different concentrations (0.01%, 0.1%, 1%) therein. This evaluation was based on the detection of the ATP levels (Crouch SPM et al.). The measurement of the ATP levels was made with a fluorimeter and by using a kit (ViaLight™ Plus Cell Proliferation and Cytotoxicity). ATP molecules are characterized as the energy coin in the intracellular energy conveyance from one biochemical molecule to another (Crouch SMP et al).

The Sideritis infusion of the invention did not present cytotoxicity on dermal fibroblasts at any concentration. On the contrary, a significant increase in the vitality of the fibroblasts (increase in ATP) to 300% (p<0.05) with a Sideritis infusion (0.01%, 0.1%, 1%) in relation to the fibroblasts without infusion was observed (barchart 1). This result is shown in the attached barchart 1.

Barchart 1. ATP levels in fibroblasts (Untreated cells) and in fibroblasts in which a Sideritis infusion has been added at three concentrations (cells+Sideritis infusion 0.01%, cells+Sideritis infusion 0.1%, cells+Sideritis infusion, 1%). * Statistically significant difference, Student's t-test (p<0.05).

Thereafter, the bioactivity of the Sideritis infusion (0.01%, 0.1%, 1%) under oxidative stress was studied. It is known that oxidative stress constitutes a major form of attack on the skin and is considered as one of the most important factors causing aging. Under oxidative stress, the formation of free radicals is activated. Free radicals are highly-active molecules which are generated as byproducts of the normal metabolism (endogenous) and environmental stress (exogenous), and are responsible for cell damages, mainly aiming at cells such as the dermal cells.

In the experimental procedure, hydrogen peroxide (H₂O₂) was used as free-radical formation agent. In particular, the experimental procedure includes the following steps:
48 h incubation of the infusion at three different concentrations (0.01%, 0.1%, 1%) on the NHDF cells
Removing the nutrient medium and washing with a phosphate/salt buffer solution (PBS) in order to avoid intramolecular reactions between the infusion and H₂O₂.
Adding a hydrogen peroxide solution (0.1, 0.5, 1 mM) for three hours.
Adding a nutrient medium
Measuring the viability of the cells based on the ATP

From the specific experimental procedure it was observed that the Sideritis infusion, at the three concentrations tested, protects the cells against oxidative stress since an increase in cell vitality (increase in ATP) was observed with a Sideritis infusion (0.01%, 0.1%, 1%) under oxidative stress conditions (addition of H₂O₂) in relation to the cells without Sideritis infusion as well as the cells under oxidative stress conditions. In the attached barchart 2 the increase in cell vitality with a Sideritis infusion 1% under conditions of oxidative stress is shown.

Barchart 2. The ATP levels in fibroblasts (Untreated cells), in fibroblasts in which a Sideritis infusion has been added (cells+Sideritis infusion 1%), fibroblasts under oxidative stress (cells+0.1 mM H₂O₂, cells+0.5 mM H₂O₂, cells+1 mM H₂O₂) and fibroblasts in which a Sideritis infusion 0.1% has been added under various oxidative stress conditions (cells+Sideritis infusion 1%+0.1 mM H₂O₂, cells+Sideritis infusion 0.1%+0.5 mM H₂O₂, cells+Sideritis infusion 0.1%+1 mM H₂O₂).

* Statistically significant difference, Student's t-test (p<0.05).

Thereafter the bioactivity of the Sideritis infusion of the invention under conditions of photooxidative stress (accelerated aging) was studied.

Ultraviolet radiation is a significant source of oxidative stress for the skin. Apart from the production of free radicals, ultraviolet radiation affects the defense enzymes of the skin against oxidation, making it even more vulnerable to permanent cell damage (acceleration of skin aging). When the skin is exposed to ultraviolet radiation for a long time without protection by sunscreens, its protection depends exclusively on the endogenous antioxidative defense systems.

In the applied experimental procedure, photooxidative stress was simulated. Therefore, a UV lamp was used at two wavelengths 312 (UVB), 365 (UVA). The UVA radiation dose was 5 J/cm² whereas the UVB radiation dose was 0.3 J/cm² for 20 minutes. The UV lamp was used as a free-radical forming agent. Specifically, the experimental procedure includes the following steps:
Incubation of the Sideritis extract 1% on NHDF cells for 48 h
Removing the nutrient medium and washing with a phosphate/salt buffer solution (PBS) in order to avoid intramolecular reactions
Adding a phosphate/salt buffer solution (PBS) and activation of the UV lamp at the two wavelengths for 20 min
Adding nutrient medium
Measuring cell viability based on the ATP.

From the specific experimental procedure it was observed that the Sideritis infusion 1% protects the cells against photooxidative stress since an increase in cell vitality (increase in ATP) was observed in relation to the cells without the infusion. This fact confirms the antioxidative capacity of the Sideritis infusion as well as its protective role against accelerated cell aging (photoaging).

Also, the variations which are caused by the influence of the 1% Sideritis infusion of the invention on the gene expression of dermal fibroblasts were studied. Therefore, real time polymerase chain reaction RT-PCR (Giulietti A et al.) was employed. From the gene study it was derived that the fibroblasts with 1% Sideritis infusion presented an overexpression (p<0.05) of the aquaporin-3 gene (AQP3) in relation to the fibroblasts without infusion, as shown in the attached barchart 3. Aquaporins are a class of proteins which form channels that facilitate massive inflow of water molecules in the cell (Zoe Draelos). Aquaporin-3 is present in major amounts in the human skin and the overexpression of the AQP3 gene has been associated with the skin moisturization (Hara-Chikruma M and Verkman A S, Fluhr J W et al., Boury-Jamot et al.).

Barchart 3. In the attached barchart 3, the expression levels of the AQP3 gene in fibroblasts in the presence or absence of a 1% Sideritis infusion (untreated cells, cells+1% Sideritis infusion, respectively) are shown.

* Statistically significant difference is shown, Student's t-test (p<0.05).

The antiaging action of the Sideritis infusion of the invention was studied based on the expression levels of the SIRT1 gene which codes for the respective protein at the mRNA level. SIRT1 is a protein, the action of which has been associated with the anti-aging protection of the dermal cells (Moreau M et al., Michishita E et al.). Overexpression of the SIRT1 gene was observed, indicating an anti-aging effect of the 1% Sideritis infusion on the human dermal fibroblasts.

Barchart 4. In the attached barchart 4, the expression levels of the SIRT1 gene in fibroblasts in the presence or absence of a 1% Sideritis infusion are depicted (untreated cells, cells+1% Sideritis infusion respectively). * Statistically significant difference is shown, Student's t-test (p<0.05).

Finally, the Sideritis infusion of the invention was applied on an in vitro three-dimensional (3D) model of reconstructed human skin, in order to investigate any irritation which it may cause on the human skin, and was evaluated as non-irritating. Based on all the above-mentioned experimental applications, the following were demonstrated:

The Sideritis infusion of the invention enhances the vitality of the human dermal fibroblasts, which was observed on three different concentrations⋅has a protective role under conditions of oxidative stress and thus a protective role against the aging of the dermal fibroblasts, an effect which was observed on three different concentrations under different conditions of oxidative stress (different H₂O₂ concentrations)⋅the Sideritis infusion of the invention has a protective role against the accelerated aging of the cells (photoaging)⋅the expression of the SIRT1 gene in fibroblasts was increased by a Sideritis infusion, confirming its anti-aging action, while an increased expression of the AQP3 gene in fibroblasts with a Sideritis infusion was observed, indicating its role on their moisturization mechanism. The infusion of the invention does not cause any irritation on the human skin.

1. An infusion obtained from a mixture of species of the Sideritis plant for topical cutaneous or other uses, wherein the mixture is formed by mixing the plants of the Sideritis family designated as Sideritis perfoliata/Sideritis scardica/Sideritis raeseri at proportions by weight from 70/25/5 to 40/50/10 respectively, with deionised water as extraction solvent which has been obtained by the reverse-osmosis method, at a proportion by weight of the mixture of the Sideritis plants to the solvent from 0.01% to 10% and is either used as such for topical cutaneous use, or as a component of cosmetic or cosmeceutical products at a proportion by weight from 0.01% to 80% in the produced final product.

2. A method of producing an infusion from a mixture of species of the Sideritis plant according to claim 1, wherein the preselected species of the plant, after having been obtained in the desired amounts, are mixed and comminuted to pieces having dimensions 1-10 mm and the mixture is placed in a sack filter having a pore diameter of 100μ, which is then placed in an extraction device with pressure and temperature control and then the solvent is added and the extraction procedure takes place under a pressure of 0 bar to 10 bar at 37° C. to 98° C., which procedure lasts from one (1) to thirty (30) minutes and after completion thereof, if the infusion is intended for direct use on the skin, the sack filter with the plants is removed from the device, the infusion is conditioned to a temperature of 20° C.-25° C. and thereafter the plants are removed from the device and passed through cartridge filters having a pore diameter of 10μ-5μ-1μ-0.2μ and the final product is directly packaged.

3. A method of producing an infusion from a mixture of species of the Sideritis plant according to claim 1, wherein if the mixture is to be a component of cosmetic and cosmeceutical products, after the completion of the extraction procedure and the removal of the sack filter with the plants from the device, the infusion is conditioned to a temperature of 50° C. to 70° C. and is filtrated through a sack filter having a pore diameter of 25μ, being used directly as an active component in the aqueous phase of cosmetic and cosmeceutical products.

4. A method of producing an infusion from a mixture of species of the Sideritis plant according to claim 1, wherein the deionised water used as extraction solvent is processed to become suitable for use and to be of quality of <=1 μS/cm at 25° C.
